(19) United States
(12) Patent Application Publication
(10) Pub. No.: US 2022/0215962 A1
(43) Pub. Date: Jul. 7, 2022

(54) IMAGE DIAGNOSIS SUPPORT DEVICE, OPERATION METHOD OF IMAGE DIAGNOSIS SUPPORT DEVICE, AND OPERATION PROGRAM OF IMAGE DIAGNOSIS SUPPORT DEVICE

(71) Applicant: FUJIFILM Corporation, Tokyo (JP)

(72) Inventor: Atsushi TACHIBANA, Tokyo (JP)

(73) Assignee: FUJIFILM Corporation, Tokyo (JP)

(21) Appl. No.: 17/699,191

(22) Filed: Mar. 21, 2022

Related U.S. Application Data
(63) Continuation of application No. PCT/JP2020/036274, filed on Sep. 25, 2020.

(30) Foreign Application Priority Data
Sep. 25, 2019 (JP) 2019-173856

(51) Int. Cl. G16H 50/20; G06T 7/00; G06F 3/01; G16H 30/40

(52) U.S. Cl. CPC G16H 50/20; G16H 30/40; G06F 3/013; G06T 7/0012

(57) ABSTRACT

An image diagnosis support device includes a display control unit that displays a medical image acquired by imaging a subject on a display unit, and a notification unit that gives, in a case in which an AI image, which is a medical image to which AI technology which is technology using artificial intelligence is applied, is displayed on the display unit as the medical image, a notification that the medical image displayed on the display unit is the AI image.

### CROSS-REFERENCE TO RELATED APPLICATIONS

This application is a continuation application of International Application No. PCT/JP2020/036274, filed on Sep. 25, 2020, which is incorporated herein by reference in its entirety. Further, this application claims priority from Japanese Patent Application No. 2019-173856, filed on Sep. 25, 2019, the disclosure of which is incorporated by reference herein in their entirety.

### BACKGROUND

### Technical Field

The present disclosure relates to an image diagnosis support device, an operation method of an image diagnosis support device, and a non-transitory storage medium storing a program.

### Related Art

In recent years, in addition to radiography apparatuses by using radiation, such as X-rays or gamma rays, due to advances in image capturing apparatuses, such as a computed tomography (CT) apparatus, an ultrasound (US) diagnostic apparatus, a magnetic resonance imaging (MM) apparatus, a positron emission tomography (PET) apparatus, and a single-photon emission computed tomography (SPECT) apparatus, it has been possible to make an image diagnosis by using a medical image having a high resolution that has a high quality. In addition, in the field of image diagnosis, the technology based on artificial intelligence (hereinafter referred to as AI) is advanced.

As the AI, for example, there is a computer-aided diagnosis (hereinafter referred to as CAD), which is a diagnosis support function by a computer. The medical image acquired by the image capturing apparatus is analyzed by the CAD, a region, a position, a volume, and the like of a lesion or the like included in the medical image are extracted, and these results are acquired as analysis results. The analysis results generated by analysis processing in this way are used for the image diagnosis by displaying the analysis results on the medical image, or storing the analysis results in a database in association with a patient name, a gender, an age, and examination information of the image capturing apparatus or the like that acquires the medical image.

In addition, based on the medical image acquired by the image capturing apparatus, a new medical image, which is an image diagnosis target, is also generated by using the AI technology. As an example, the technology has been proposed in which a slice thickness of a CT image acquired by a CT apparatus is virtually thinned by using the AI technology (see JP2008-110098A). This technology is the technology of virtually generating the CT image having the slice thickness of about 1 mm, for example, based on the CT image having the slice thickness of about 5 mm set at the time of imaging. By virtually thinning the slice thickness, it is possible to improve the visibility of bones or improve the image quality in a case in which the image is three-dimensionally displayed.

In this way, information more useful for the image diagnosis may be able to be obtained by applying the image analysis technology by using the AI technology and the image generation technology by using the AI technology to the medical image captured by the image capturing apparatus.

Here, a medical image to which the AI technology is applied to a medical image captured by an image capturing apparatus is referred to as an AI image. In addition, in the medical image captured by the image capturing apparatus, a medical image to which the AI technology is not applied is referred to as a non-AI image in comparison with the AI image. As described above, the AI image includes the medical image obtained by analyzing the non-AI image by the AI technology and adding the analysis results obtained by the analysis to the non-AI image, which is an analysis target, and the medical image, which is newly generated separately from the original non-AI image, by applying the AI technology to the non-AI image.

In a case in which the AI image is used, the information useful for diagnosis is obtained, the number of situations in which the AI image is used has been increased in the medical field in which the medical image diagnosis is made. As the medical image used for the final definitive diagnosis of the patient, the AI image and the non-AI image are mixed. On the other hand, since the AI technology has insufficient accumulation of reliability at least at a current stage as compared to a determination of a doctor, it is currently unacceptable to rely on the AI image for all diagnosis evidence.

Under these circumstances, it is important to clearly distinguish whether the medical image used as the evidence of diagnosis is the AI image or the non-AI image. However, it may be difficult to distinguish between the AI image and the non-AI image only by looking at the image. Therefore, in a case in which the medical image is displayed on an image display terminal used by the doctor, it has been required to be able to easily distinguish whether or not the displayed medical image is the AI image.

### SUMMARY

The present disclosure has been made in view of the circumstances described above, and is to provide an image diagnosis support device, an operation method of an image diagnosis support device, and a non-transitory storage medium storing a program for an image diagnosis support processing that can easily distinguish whether or not a medical image displayed on a display unit is an AI image.

A first aspect of the present disclosure relates to an image diagnosis support device comprising a display control unit that displays a medical image acquired by imaging a subject on a display unit, and a notification unit that gives, in a case in which an AI image, which is a medical image to which AI technology which is technology using artificial intelligence is applied, is displayed on the display unit as the medical image, a notification that the medical image displayed on the display unit is the AI image.

Note that, in the image diagnosis support device according to the present aspect, the AI image may be a medical image newly generated separately from the medical image by applying the AI technology to the medical image.

In addition, in the image diagnosis support device according to the present aspect, the AI image may be a medical image obtained by adding, to the medical image, an image analysis result obtained by performing an image analysis by using the AI technology based on the medical image.

In addition, in the image diagnosis support device according to the present aspect, the notification unit may display an AI flag indicating that the AI technology is applied to the AI image.

In addition, the image diagnosis support device according to the present aspect may further comprise the determination unit that determines whether or not the medical image displayed on the display unit is the AI image.

In addition, in the image diagnosis support device according to the present aspect, accessory information of the medical image may include information indicating whether or not the AI technology is applied, and the determination unit may determine whether or not the medical image is the AI image based on the accessory information.

In addition, the image diagnosis support device according to the present aspect may further comprise a browsing detection unit that detects whether or not a user browses the AI image, and a recording control unit that performs a control of recording a browsing history indicating that the AI image is browsed, based on a detection result of the browsing detection unit.

In addition, in the image diagnosis support device according to the present aspect, the browsing detection unit may detect that the AI image is browsed in a case in which the AI image, which is not displayed on the display unit, is displayed on the display unit.

In addition, in the image diagnosis support device according to the present aspect, the browsing detection unit may detect that the AI image is browsed in a case in which a display instruction for displaying the AI image, which is not displayed, on the display unit is input.

In addition, the image diagnosis support device according to the present aspect may further comprise a gaze detection unit that detects a gaze of the user, in which the browsing detection unit detects that the AI image is browsed in a case in which the gaze detection unit detects that the gaze of the user is directed to the AI image displayed on the display unit.

In addition, in the image diagnosis support device according to the present aspect, the recording control unit may further perform a control of recording a use history indicating that the AI image is used for an image diagnosis, based on an operation of the user.

In addition, the image diagnosis support device according to the present aspect may further comprise a warning unit that issues, in a case in which a report related to the image diagnosis is created in a state in which the use history is not present even though the browsing history is present, a warning that the use history is not present at least before creation of the report is terminated.

A second aspect of the present disclosure relates to an operation method of an image diagnosis support device, the method comprising displaying a medical image acquired by imaging a subject on a display unit, and giving, in a case in which an AI image, which is a medical image to which AI technology which is technology using artificial intelligence is applied, is displayed on the display unit as the medical image, a notification that the medical image displayed on the display unit is the AI image.

A third aspect of the present disclosure relates to a non-transitory storage medium storing a program that causes a computer to perform an image diagnosis support processing, the image diagnosis support processing includes displaying a medical image acquired by imaging a subject on a display unit, and giving, in a case in which an AI image, which is a medical image to which AI technology which is technology using artificial intelligence is applied, is displayed on the display unit as the medical image, a notification that the medical image displayed on the display unit is the AI image.

Note that a fourth aspect of the present disclosure relates to an image diagnosis support device comprising a memory that stores a command to be executed by a computer, and a processor configured to execute the stored command, in which the processor is configured to display a medical image acquired by imaging a subject on a display unit, and to give, in a case in which an AI image, which is a medical image to which AI technology which is technology using artificial intelligence is applied, is displayed on the display unit as the medical image, a notification that the medical image displayed on the display unit is the AI image.

According to the aspects of the present disclosure, it is possible to easily distinguish whether or not the medical image displayed on the display unit is the AI image.

### BRIEF DESCRIPTION OF THE DRAWINGS

FIG. 1 is a diagram showing a schematic configuration of a diagnosis support system to which an image diagnosis support device of one embodiment of the present disclosure is applied.

FIG. 2 is a diagram for describing an AI image and a non-AI image.

FIG. 3 is a diagram for describing the AI image.

FIG. 4 is a schematic block diagram showing a configuration of the image diagnosis support device according to the embodiment of the present disclosure.

FIG. 5 is a functional block diagram of the image diagnosis support device according to a first embodiment.

FIG. 6 is a diagram showing an example of display on a display screen of a display unit according to the first embodiment.

FIG. 7 is a flowchart showing processing performed in the first embodiment.

FIG. 8 is a functional block diagram of an image diagnosis support device according to a second embodiment.

FIG. 9 is a diagram showing an example of display (AI image non-display) on the display screen of the display unit according to the second embodiment.

FIG. 10 is a diagram showing an example of display (AI image display) of the display screen of the display unit according to the second embodiment.

FIG. 11 is a functional block diagram of an image diagnosis support device according to a third embodiment.

FIG. 12 is a diagram for describing a gaze detection unit.

FIG. 13 is a functional block diagram of an image diagnosis support device according to a fourth embodiment.

FIG. 14 is a diagram showing an example of the display screen of the display unit according to the fourth embodiment.

FIG. 15 is a diagram showing an example of display on the second display screen of the display unit according to the fourth embodiment.

FIG. 16 is a flowchart showing processing performed in the fourth embodiment (part 1).

FIG. 17 is a flowchart showing processing performed in the fourth embodiment (part 2).

### DETAILED DESCRIPTION

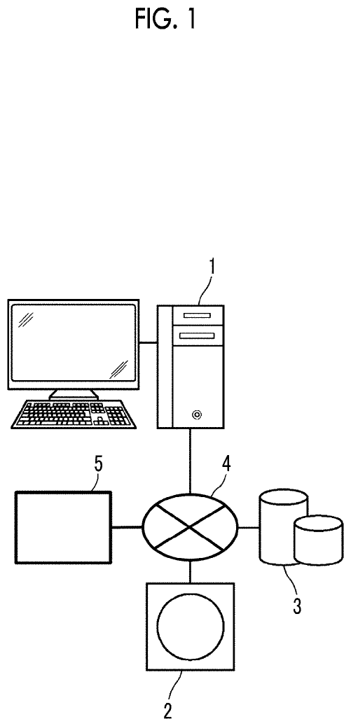

In the following, a first embodiment of the present disclosure will be described with reference to the drawings. FIG. 1 is a diagram showing a schematic configuration of a diagnosis support system to which an image diagnosis support device of the embodiment of the present disclosure is applied. As shown in FIG. 1, in the diagnosis support system, an image diagnosis support device 1 according to the present embodiment, an image capturing apparatus 2, an image storage server 3, and an image processing unit 5 are connected via a network 4 in a communicable state.

The image capturing apparatus 2 is an apparatus that images a site, which is a diagnosis target, of a patient, which is an example of a subject, and generates an image representing the site. Specifically, in addition to a radiography apparatus using radiation, such as X-rays, a CT apparatus, an ultrasound diagnostic apparatus, an MRI apparatus, a PET apparatus, and an SPECT apparatus. A medical image, such as a two-dimensional image and a three-dimensional image, captured by the image capturing apparatus 2 is transmitted to and stored in the image storage server 3.

In the present disclosure, the three-dimensional image is a set of a plurality of slice images (tomographic images) output by a tomography apparatus, such as the CT apparatus or the MRI apparatus, and is also called volume data. In addition, in the present disclosure, the volume data acquired by one imaging is referred to as an “image group”. In addition, in the present disclosure, the two-dimensional image is each slice image included in the image group, an X-ray image acquired by simple X-ray imaging using, for example, the radiography apparatus, and the like. In the present disclosure, the three-dimensional image and the two-dimensional image are examples of a medical image.

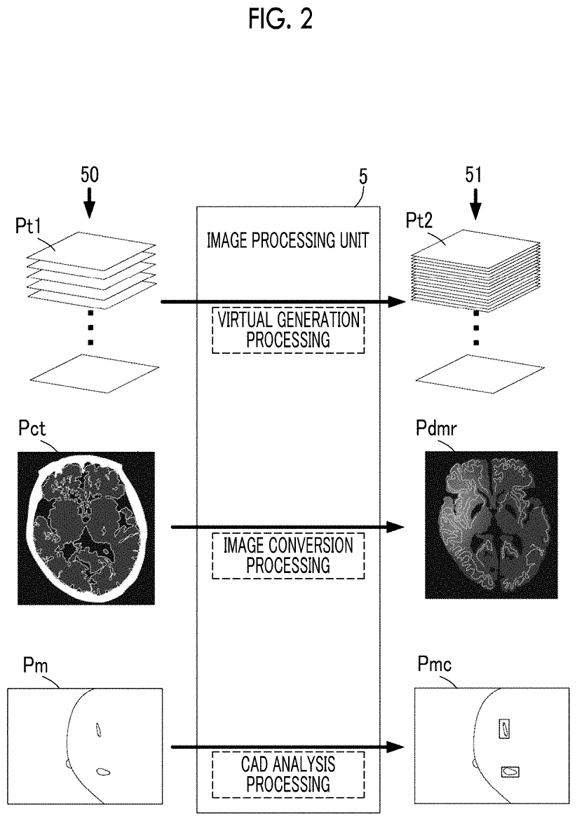

The image processing unit 5 performs various pieces of processing on the medical image captured by the image capturing apparatus 2 by using the AI technology, which is the technology using artificial intelligence. Note that in the technology of the present disclosure, the medical image, which is obtained by performing various pieces of processing using the AI technology by the image processing unit 5 to the medical image captured by the image capturing apparatus 2, is referred to as an AI image 51. In addition, in the medical image captured by the image capturing apparatus 2, the medical image to which the AI technology is not applied is referred to as a non-AI image 50 in comparison with the AI image. FIG. 2 is a diagram for describing the AI image 51 and the non-AI image 50.

As shown in FIG. 2, in a case in which the non-AI image 50 is input, the image processing unit 5 performs various pieces of processing by using the AI technology on the input non-AI image, and outputs the AI image 51 to which the AI technology is applied. For example, the plurality of slice images output by the tomography apparatus, such as the CT apparatus and the Mill apparatus, are input to the image processing unit 5 as the non-AI images. The image processing unit 5 performs virtual generation processing on the input non-AI image 50, that is, the plurality of slice images, virtually generates the AI image 51 which is a slice image having a slice thickness t2 thinner than a slice thickness tl of the input slice image, and outputs the generated AI image 51.

Here, the virtual generation processing will be described. In the present embodiment, in the virtual generation processing, a first discriminator which is machine-learned by using learning information including a plurality of data sets of a pair of the plurality of slice images (hereinafter also referred to as a first image group Pt1) having the slice thickness t1 and the plurality of slice images (second image group Pt2) having the slice thickness t2, which are actually captured by the tomography apparatus, such as the CT apparatus and MRI apparatus is used. The first discriminator is learned such that the second image group Pt2 is output in a case in which the first image group Pt1 is input. By using the first discriminator learned in this way, the image processing unit 5 can virtually generate the second image group Pt2 (AI image 51) having the slice thickness t2 from the first image group Pt1 (non-AI image 50) having the slice thickness t1.

In addition, for example, one of a plurality of CT tomographic images Pct output by the CT apparatus is input to the image processing unit 5 as the non-AI image 50. The image processing unit 5 performs image conversion processing on the input non-AI image 50, that is, the CT tomographic image Pct, and converts the CT tomographic image Pct into a virtual MR tomographic image Pdmr just like an MR tomographic image Pmr captured by the MRI apparatus.

In the present embodiment, in the image conversion processing, a second discriminator which is machine-learned by using learning information including a plurality of data sets of a pair of the CT tomographic image Pct output by the CT apparatus and the MR tomographic image Pmr output by the MRI apparatus is used. The second discriminator is learned to output the MR tomographic image Pmr in a case in which the CT tomographic image Pct is input. By using the second discriminator learned in this way, the image processing unit 5 can convert the CT tomographic image Pct (non-AI image 50) into the virtual MR tomographic image Pdmr (AI image 51).

As described above, the image processing executed by the image processing unit 5 includes image processing of newly generating the AI image 51, which is the medical image separately from the original non-AI image 50, by applying the AI technology to the non-AI image 50.

In addition, the AI image 51 includes the following medical images, as well as the image processing of generating the new AI image 51 based on the non-AI image 50. For example, a breast image Pm acquired by simple imaging performed by a mammography apparatus, which is an example of the radiography apparatus, is input to the image processing unit 5 as the non-AI image 50. The image processing unit 5 analyzes the input non-AI image 50, that is, the breast image Pm by CAD, extracts a size, a position, a volume, and the like of a region of interest, such as a lesion, included in the breast image Pm, and acquires the extracted results as the analysis results. The AI technology using a machine learning model, such as a neural network, is applied to the CAD analysis processing in the present embodiment. The image processing unit 5 generates a marked breast image Pmc having a frame surrounding the region of interest on the breast image Pm based on the analysis results generated by the CAD analysis processing.

In this way, the image processing unit 5 analyzes the non-AI image 50 by the AI technology, and generates the image obtained by adding the analysis results obtained by the CAD analysis processing to the non-AI image 50, which is the analysis target, as the AI image 51.

Summarizing the above description, in the technology of the present disclosure, the medical image newly generated separately from the original non-AI image 50 by applying the AI technology to the non-AI image 50 is also the AI image 51, and the medical image obtained by adding the image analysis results obtained by performing the image analysis by using the AI technology based on the non-AI image 50 to the non-AI image 50, which is the analysis target, is also the AI image 51. Note that in the present disclosure, the image generated by performing the analysis on the AI image 51 newly generated separately from the original non-AI image 50, and adding the analysis results obtained by the analysis to the AI image 51, which is the analysis target, is also the AI image 51.

The image storage server 3 is a computer that stores and manages various data, and comprises a large capacity external storage device and software for database management. The image storage server 3 performs communication with other devices via the wired or wireless network 4 to transmit and receive image data. Specifically, the image storage server 3 acquires various data including the image data of an examination image generated by the image capturing apparatus 2 via the network, and stores and manages the data in a recording medium, such as the large capacity external storage device. Note that a storage format of the image data and the communication between the devices via the network 4 are based on a protocol, such as digital imaging and communication in medicine (DICOM).

In the present embodiment, the image storage server 3 stores the examination image for each patient. As the examination image stored for each patient, for example, there are a plurality of the examination images acquired by a plurality of examinations performed on the same patient. These examination images are stored for each examination. In addition, even in one examination for the same patient, there are usually the plurality of examination images. As the plurality of examination images acquired in one examination, for example, in a case of the breast examination, there are the examination images having different imaging conditions, such as an MLO image obtained by MLO imaging and a CC image obtained by CC imaging. In addition, the same type of examination may be performed a plurality of times on different examination dates, such as follow-up. The plurality of examinations having different examination dates are treated as different examinations, for example, and the plurality of examination images having different examination dates are stored for each examination date. In this way, the image storage server 3 stores the latest (current) examination images and the past examination images for the same type of examination, in addition to the different types of examination images performed on the same patient.

In the present embodiment, the examination image immediately after being acquired by the examination will be described as the non-AI image 50 to which the AI technology is not applied. In addition, the image storage server 3 also stores, in addition to the examination image which is the non-AI image 50, the AI image 51 generated by the image processing unit 5 performing the various pieces of processing described above on the examination image. That is, the non-AI image 50 and the AI image 51, which are examples of the medical image, are stored in the image storage server 3.

In addition, each medical image includes accessory information, such as a DICOM tag, in addition to an image main body. Examples of the accessory information include information, such as an image identification (ID) for identifying an individual image, a patient ID for identifying the subject, an examination ID for identifying the examination, the examination date when the examination image, which is the original image before the AI technology is applied, is generated, an examination time point, a type of the image capturing apparatus 2 used in the examination to acquire the examination image, patient information, such as the patient name, the age, and the gender, examination site (imaging site), and an imaging condition (whether or not a contrast medium is used or the radiation dose). In addition, the accessory information also includes information, such as a CAD result in a case in which the CAD processing is performed.

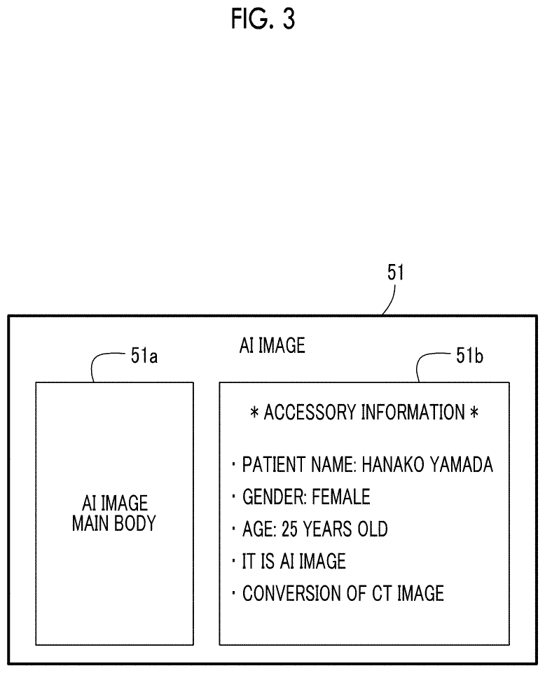

In the present embodiment, it is premised that the accessory information included in the AI image 51 includes identification information indicating that the AI image is the AI image. FIG. 3 is a diagram for describing the AI image. As shown in FIG. 3, the AI image 51 is configured by an AI image main body 51a and accessory information 51b. As an example, the accessory information 51b includes information, such as “Hanako Yamada” as the patient name, “female” as the gender, “25 years old” as the age, “it is the AI image” as whether or not it is the AI image, and “conversion of the CT image” as an image processing method. The information described as “it is the AI image” in FIG. 3 is the identification information indicating that it is the AI image. The identification information of the AI image may be text information, but is actually recorded in the form of, for example, a flag or a code.

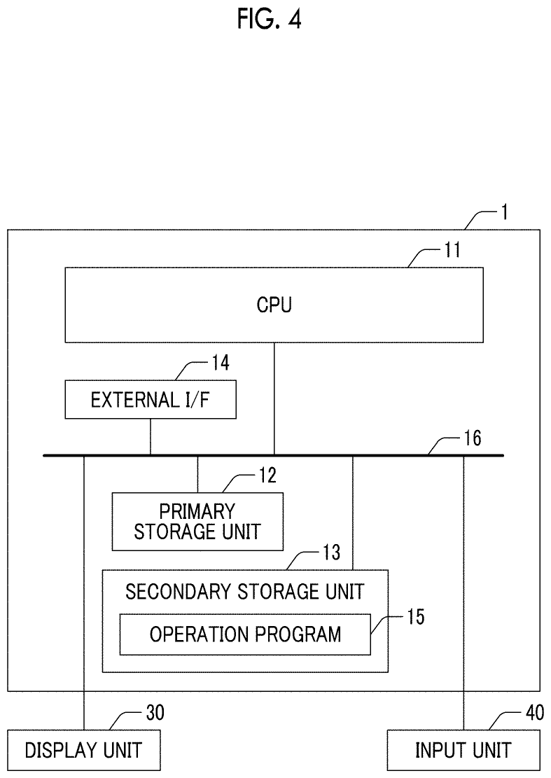

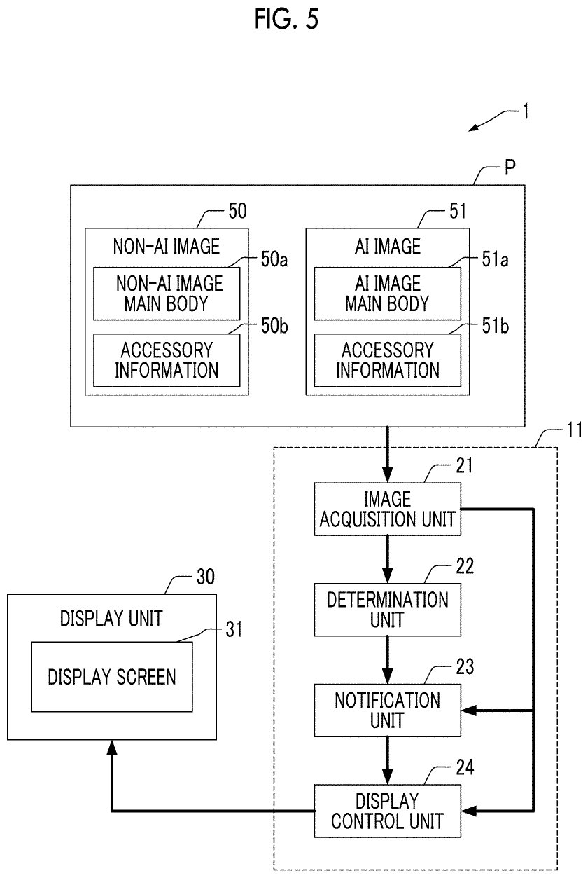

Next, a configuration of the image diagnosis support device 1 will be described. FIG. 4 is a block diagram showing the configuration of the image diagnosis support device 1 according to the embodiment of the present disclosure, and FIG. 5 is a functional block diagram of the image diagnosis support device 1 according to the first embodiment.

The image diagnosis support device 1 is configured by a computer comprising a central processing unit (CPU) 11, a primary storage unit 12, a secondary storage unit 13, an external interface (I/F) 14, and the like. The CPU 11 controls the whole image diagnosis support device 1. The primary storage unit 12 is a volatile memory used as a work area or the like when various programs are executed. Examples of the primary storage unit 12 include a random access memory (RAM). The secondary storage unit 13 is a non-volatile memory in which various programs, various parameters, and the like are stored in advance, and one embodiment of an operation program 15 of the image diagnosis support device 1 of the present disclosure is installed. Examples of the secondary storage unit 13 include a hard disk drive, a solid state drive, and a flash memory.

The operation program 15 is distributed in a state of being recorded on a storage medium, such as a digital versatile disc (DVD) or a compact disc read only memory (CD-ROM), and is installed in the computer from the storage medium. Alternatively, the operation program 15 may be stored in a storage device or a network storage of a server computer connected to the network in a state of being accessible from the outside, downloaded to the computer in response to an external request, and then installed.

By executing the operation program 15 by the CPU 11, the CPU 11 functions as an image acquisition unit 21, a determination unit 22, a notification unit 23, and a display control unit 24 shown in FIG. 5.

The external I/F 14 controls the transmission and reception of various pieces of information between the image diagnosis support device 1 and the image storage server 3. The CPU 11, the primary storage unit 12, the secondary storage unit 13, and the external I/F 14 are connected to a bus line 16, which is a common route for exchanging data.

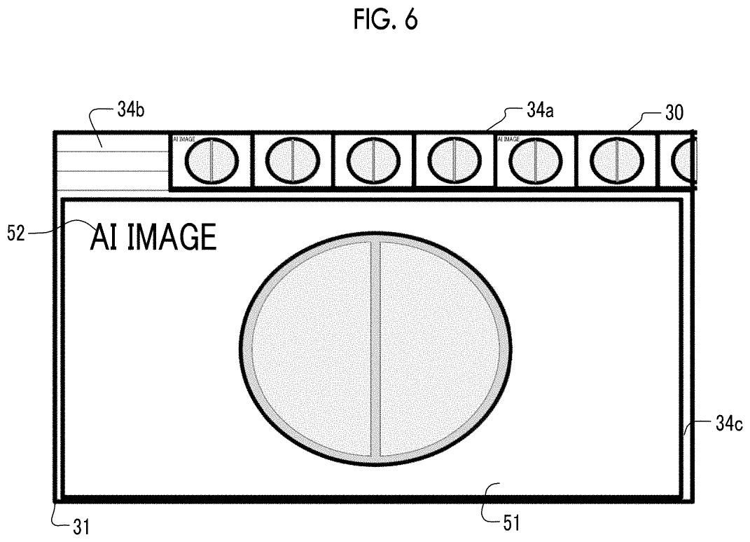

In addition, a display unit 30 and an input unit 40 are also connected to the bus line 16. The display unit 30 is configured by, for example, a liquid crystal display. As will be described below, the display unit 30 displays a display screen (see reference numeral 31 in FIG. 6) on which various regions including an image display region are displayed. Note that the display unit 30 may be configured by a touch panel and may also be used as the input unit 40. The input unit 40 comprises a mouse, a keyboard, and the like, and inputs various settings by the user. The input unit 40 according to the present embodiment functions as a mouse for inputting a selection operation of the medical image to be displayed on a display screen 31 and a mouse for inputting various operations on the medical image displayed on the display screen.

The image acquisition unit 21 acquires the medical image from the image storage server 3 via the external I/F 14. The image acquisition unit 21 acquires the medical image selected by the user by operating the input unit 40. In the present embodiment, for example, as shown in FIG. 5, the image acquisition unit 21 acquires the examination images acquired by the image capturing apparatus 2, which are the non-AI image 50 to which the AI technology is not applied and the AI image 51 obtained by applying the AI technology to the non-AI image 50. The medical image acquired by the image acquisition unit 21 is displayed on the display screen 31 of the display unit 30.

In the following, the functions of the image diagnosis support device 1 will be described based on the functional blocks shown in FIG. 5 and a display screen example shown in FIG. 6. FIG. 6 is a diagram showing an example of the display of the display screen 31 of the display unit 30 according to the present embodiment. The display screen 31 is an example of a graphical user interface (GUI) that functions as an operation screen on which the examination image and various operation units are displayed.

As shown in FIG. 6, a thumbnail image display region 34a in which a thumbnail image obtained by reducing the medical image is displayed is provided in the upper right of the display screen 31. In addition, in the upper left of the display screen 31, although shown briefly, a selection region 34b is provided in which a patient list on which the patient ID is displayed and an examination list of examinations performed on each patient are displayed in a selectable manner. In addition, below the thumbnail image display region 34a and the selection region 34b, an image display region 34c on which the medical image is displayed is provided.

For example, in a case in which the user selects the patient ID of the patient to be interpreted from the patient list, the examination list of the selected patient is displayed. In a case in which the user selects the examination including the examination image to be displayed from the displayed examination list, the examination image acquired by the selected examination, that is, the thumbnail image of the non-AI image 50 is displayed in the thumbnail image display region 34a. In addition, in the examination selected by the user, various pieces of processing are performed on the examination image by the image processing unit 5, and in a case in which the AI image 51 to which the AI technology is applied is present, the thumbnail image of the AI image 51 is also displayed in the thumbnail image display region 34a. That is, in the thumbnail image display region 34a, the thumbnail image of the medical image including at least one of the non-AI image 50 or the AI image 51 is displayed.

In a case in which the user selects the thumbnail image corresponding to the medical image to be interpreted from a plurality of the thumbnail images displayed in the thumbnail image display region 34a, the image acquisition unit 21 acquires the medical image corresponding to the selected thumbnail image as the medical image selected by the user.

The determination unit 22 determines whether the medical image acquired by the image acquisition unit 21 is the non-AI image 50 or the AI image 51. As a determination method, as described above, a determination is made based on each medical image, that is, the accessory information 50b and 51b included in the non-AI image 50 and the AI image 51, respectively. Specifically, the determination is made based on the information on whether or not the AI image, which is included in the accessory information 50b and 51b. The determination unit 22 determines that the medical image is the AI image 51 in a case in which information on “it is the AI image” is included in the accessory information 50b and 51b.

In a case in which the determination unit 22 determines that the medical image acquired by the image acquisition unit 21 is the AI image 51, the notification unit 23 gives a notification that the medical image displayed on the display unit is the AI image 51 in a case in which the AI image 51 is displayed on the display screen 31 of the display unit 30. Specifically, as a flag indicating that the AI technology is applied to the AI image 51 to be displayed, in FIG. 6, as an example, an AI flag 52 indicated by text information, such as “AI image”, is displayed on the display control unit 24.

The display control unit 24 displays the medical image acquired by the image acquisition unit 21 on the display screen 31. In addition, in the present embodiment, in a case in which the display control unit 24 further displays the AI image 51 on the display screen 31 based on the instruction from the notification unit 23, the AI flag 52 is displayed on the AI image 51 as shown in FIG. 6.

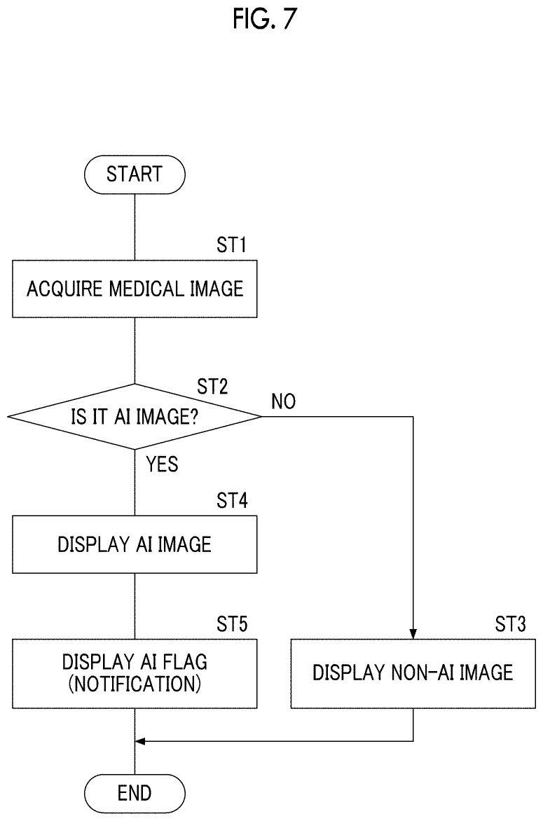

Then, processing performed in the present embodiment will be described. FIG. 7 is a flowchart showing the processing performed in the first embodiment of the present disclosure.

First, the image acquisition unit 21 acquires the medical image (step ST1). Specifically, as described above, the user selects the patient name to be interpreted from the patient list by using the input unit 40, and selects a desired examination from the examination list of the selected patient. As a result, the thumbnail image of the medical image acquired by the selected examination is displayed in the thumbnail image display region 34a. In the present embodiment, as an example, the thumbnail image includes the thumbnail images of the non-AI image 50 and the AI image 51.

In a case in which the user selects the thumbnail image to be displayed from the plurality of thumbnail images displayed in the thumbnail image display region 34a, the image acquisition unit 21 searches for the medical image corresponding to the selected thumbnail image in the image storage server 3, and acquires the searched medical image. In the present embodiment, an example will be described in which the thumbnail image of the AI image 51 is selected as the thumbnail image selected by the user. The image acquisition unit 21 acquires the AI image corresponding to the selected thumbnail image as the medical image.

Then, the determination unit 22 determines whether or not the medical image acquired by the image acquisition unit 21 is the AI image 51 (step ST2). Specifically, the determination unit 22 examines the accessory information (see FIG. 3) added to the medical image, and determines whether or not the medical image is the AI image 51.

In a case in which step ST2 is denied (step ST2: NO), since the acquired medical image is not the AI image 51, that is, the non-AI image 50, the display control unit 24 displays the acquired medical image, that is, the non-AI image 50 on the display screen 31 (step ST3), and the CPU 11 terminates the processing.

On the other hand, in a case in which step ST2 is affirmed (step ST2: YES), the display control unit 24 displays the acquired medical image, that is, the AI image 51 on the display screen 31 (step ST4). Then, the notification unit 23 causes the display control unit 24 to display the AI flag 52 (see FIG. 6) indicating that the AI technology is applied to the displayed AI image 51 (step ST5), and the CPU 11 terminates the processing. Note that, in the present embodiment, displaying the AI flag 52 is an example of giving the notification that it is the AI image.

In the field of the image diagnosis, information more useful for the image diagnosis may be able to be obtained by applying the image analysis technology by using the AI technology, the image generation technology, and the like by using the AI technology to the medical image captured by the image capturing apparatus 2. In a case in which the AI image 51 is used, the information useful for diagnosis is obtained, the number of situations in which the AI image 51 is used has been increased in the medical field in which the image diagnosis is made. As the medical image used for the final definitive diagnosis of the patient, the AI image 51 and the non-AI image 50 are mixed. On the other hand, since the AI technology has insufficient accumulation of reliability at least at a current stage as compared to a determination of a doctor, it is currently unacceptable to rely on the AI image for all diagnosis evidence. Under these circumstances, it is important to clearly distinguish whether the medical image used as the evidence of diagnosis is the AI image or the non-AI image.

In the present embodiment, in a case in which the AI image 51 is displayed as the medical image on the display screen 31, the notification that the displayed medical image is the AI image 51 is given. As a result, even in a case in which it is difficult to distinguish between the AI image 51 and the non-AI image 50 only by looking at the image, it is possible to easily distinguish whether or not the medical image displayed on the display screen 31 of the display unit 30 is the AI image.

Note that, in the first embodiment, in the flowchart of FIG. 7, the display control unit 24 displays the AI image 51 on the display screen 31 (step ST4), and then the notification unit 23 gives the notification of the AI flag 52 (step ST5), but the technology of the present disclosure is not limited to this. For example, the notification unit 23 may first give the notification of the AI flag 52 (step ST5), and then the display control unit 24 may display the AI image 51.

In addition, in the first embodiment, as shown in FIG. 6, the notification unit 23 displays the text information “AI image” as the AI flag 52 on the upper left of the AI image 51, but the technology of the present disclosure is not limited to this. A display position of the AI flag 52 may be any position in the AI image 51. In addition, the AI flag 52 may be displayed around the AI image 51 instead of in the AI image 51. In addition, the display position of the AI flag 52 does not have to be around the AI image 51 as long as a correspondence between the AI image 51 and the AI flag 52 can be understood. For example, even in a case in which the AI image 51 and the AI flag 52 are positioned at positions separated from each other on the display screen 31, the correspondence is shown by connecting the AI image 51 and the AI flag 52 with a leader line or the like. In addition, the AI flag 52 may be displayed at a position separated from the AI image 51, and both an outer frame of the AI image 51 and the AI flag 52 may be turned on and off at the same timing. Also in this method, it is possible to indicate the correspondence between the AI image 51 and the AI flag 52.

In addition, the text information is used as the AI flag 52, a noun, such as the “AI image”, may be used, or a sentence, such as “this image is the AI image” may be used. In this way, any text information may be used as long as it can transmit that it is the AI image 51. In addition, the AI flag 52 does not have to be the text, but may be a figure, a symbol, a pattern, or the like recognized as a flag indicating the AI. In addition, the means of notification is not limited to display. For example, a voice “this image is the AI image” may be output.

In addition, in the first embodiment, the determination unit 22 searches for the accessory information in a case in which it is determined whether or not the medical image acquired by the image acquisition unit 21, that is, the medical image to be displayed is the AI image, but the technology of the present disclosure is not limited to this. For example, in a case in which the determination unit 22 can determine whether or not the AI technology is applied by performing the image analysis on the medical image, it may be determined whether or not the medical image is the AI image by the image analysis. In addition, in a case in which the analysis results are added to the medical image, in a case in which it is possible to determine whether or not the AI technology is applied by examining the added analysis results by the determination unit 22, it may be determined whether or not the medical image is the AI image by the image analysis.

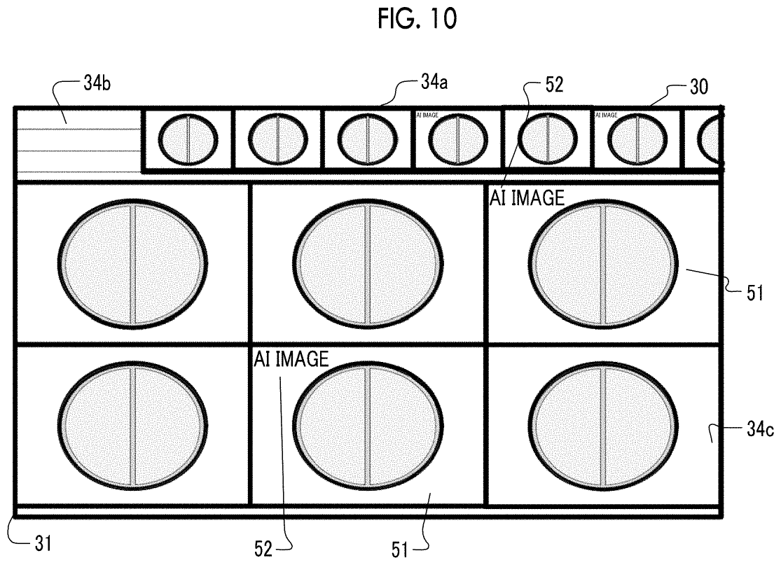

In addition, in the first embodiment, as shown in FIG. 6, the example has been described in which the medical image displayed on the display screen 31 is one, but the technology of the present disclosure is not limited to this, and a plurality of medical images may be displayed on the display screen 31. In a case in which the plurality of medical images are displayed, for example, the display screen 31 is divided into a plurality of regions based on the number of medical images acquired by the image acquisition unit 21, and the acquired medical images are displayed on the divided regions. In a case in which the AI image 51 and the non-AI image 50 are mixed in the acquired medical image, that is, the medical image to be displayed, the AI flag 52 is displayed only on the AI image 51 (see FIG. 10). Note that a method of division (size, number, shape, and the like of each region) on the display screen 31 can be optionally set by the user.

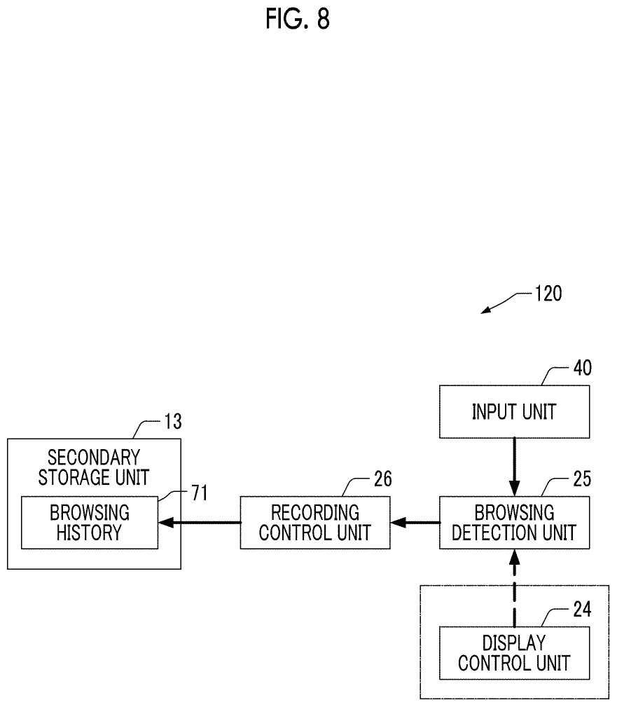

Next, a second embodiment of the present disclosure will be described. FIG. 8 is a functional block diagram of an image diagnosis support device 120 according to the second embodiment. In the image diagnosis support device 120 according to the second embodiment shown in FIG. 8, the CPU 11 of the image diagnosis support device 1 according to the first embodiment shown in FIG. 5 further has the functions of a browsing detection unit 25 and a recording control unit 26.

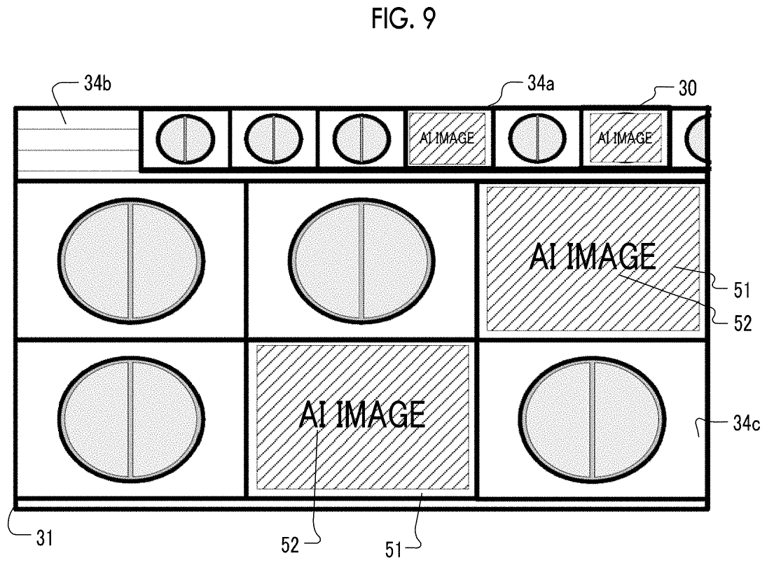

As shown in FIG. 8, the image diagnosis support device 120 according to the second embodiment comprises the browsing detection unit 25 and the recording control unit 26. The browsing detection unit 25 detects whether or not the user browses the AI image 51. FIG. 9 is a diagram showing an example of display (AI image non-display) on the display screen of the display unit according to the second embodiment, and FIG. 10 is a diagram showing an example of display (AI image display) of the display screen of the display unit according to the second embodiment.

In the present embodiment, as shown in FIG. 9, the display control unit 24 divides the display screen 31 into regions of three columns and two rows, and displays six medical images acquired by the image acquisition unit 21 in the divided regions. In the present embodiment, for example, the determination unit 22 determines that two of the six medical images are the AI images 51. In this case, in a case in which the two medical images determined as the AI image 51 are displayed on the display screen 31, the display control unit 24 displays the subject in the AI image 51 in an invisible manner, and displays the AI flag 52 in a visible manner. That is, the display control unit 24 does not display the AI image 51 while displaying the AI flag 52. Specifically, as shown in FIG. 10, the display control unit 24 does not display an image content in the display region of the AI image 51 by using hatching or the like, and displays the AI flag 52 on the display region. Note that the same processing is performed to the thumbnail image corresponding to the AI image 51.

The display control unit 24 displays the AI image 51 in a visible manner in a case in which the AI flag 52 of the AI image 51, which is not displayed, is clicked by the user operating the input unit (mouse) 40. Then, after the AI image 51 is displayed, the display control unit 24 displays the AI flag 52 on the displayed AI image 51 as shown in FIG. 10.

In the present embodiment, the browsing detection unit 25 detects that the AI image 51 is browsed in a case in which the AI flag 52 is clicked in a state in which the AI image 51 shown in FIG. 9 is not displayed. Note that the click operation of the AI flag 52 by the user corresponds to the input of a display instruction for displaying the AI image 51, which is not displayed, according to the present disclosure on the display screen 31.

As shown in FIG. 8, the recording control unit 26 stores a browsing history 71 indicating that the AI image 51 is browsed in the secondary storage unit 13 based on the detection result of the browsing detection unit 25. Specifically, the recording control unit 26 records the browsing history 71 in the secondary storage unit 13 in association with the browsed AI image 51, that is, the image ID of the AI image 51 for which the display instruction is given.

In the present embodiment, the browsing detection unit 25 detects that the user browses the AI image 51 in a case in which the display instruction for displaying the AI image 51, which is not displayed, on the display screen 31 of the display unit 30 is input (AI flag 52 is clicked). Further, the recording control unit 26 performs a control of recording the browsing history 71 based on the detection result of the browsing detection unit 25, that is, in a case in which the AI flag 52 is clicked. As a result, it is possible to leave the evidence that the doctor looks at the AI image.

Note that, in the second embodiment, the click operation has been described as an example of inputting the display instruction for displaying the AI image 51, which is not displayed, on the display screen 31, but the technology of the present disclosure is not limited to this. For example, in a case in which the display unit 30 is configured by the touch panel, the user may tap the region of the AI image 51, which is not displayed, or the AI flag 52.

In addition, in the second embodiment, the browsing detection unit 25 detects that the AI image 51 is browsed when the AI flag 52 is clicked, but the technology of the present disclosure is not limited to this. For example, in a case in which the display control unit 24 displays the AI image 51 (see FIG. 9), which is not displayed, on the display screen 31 of the display unit 30 (see FIG. 10), the browsing detection unit 25 may detect that the AI image 51 is browsed. In this case, in FIG. 8, the browsing detection unit 25 does not need to input from the input unit 40, and detects that the AI image 51 is browsed based on the input from the display control unit 24 surrounded by an alternate long and short dash line. That is, the trigger for displaying the AI image 51, which is not displayed, is not necessarily limited to the display instruction from the input unit 40. In a case in which the display control unit 24 performs a display control of the display screen 31, the AI image 51 may be displayed regardless of the operation instruction of the user. In that case, the display control unit 24 transmits, to the browsing detection unit 25, the fact that the processing of displaying the AI image 51, which is not displayed, is executed. As a result, the browsing detection unit 25 detects that the AI image 51 is browsed.

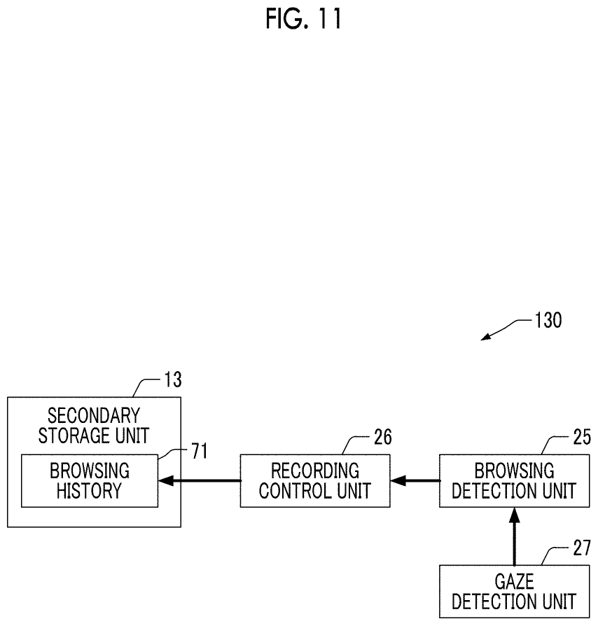

Next, a third embodiment of the present disclosure will be described. FIG. 11 is a functional block diagram of an image diagnosis support device 130 according to the third embodiment. In the image diagnosis support device 130 according to the third embodiment shown in FIG. 11, the CPU 11 of the image diagnosis support device 1 according to the first embodiment shown in FIG. 5 further has the functions of the browsing detection unit 25, the recording control unit 26, and a gaze detection unit 27. Note that the functions of the browsing detection unit 25 and the recording control unit 26 are the same as those in the second embodiment, and thus the description thereof is omitted here.

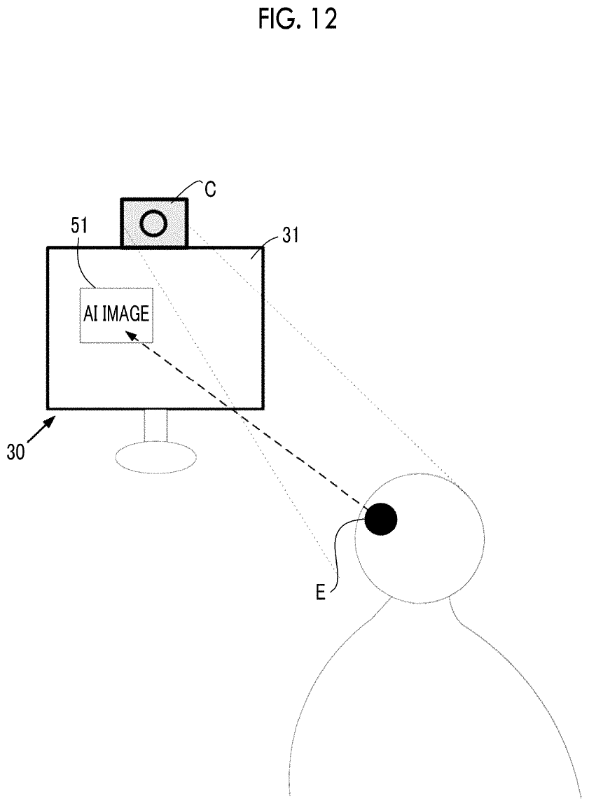

In the second embodiment, the browsing detection unit 25 detects that the AI image 51 is browsed in a case in which the AI flag 52 is displayed, but in the present embodiment, it is detected that the AI image 51 is browsed in a case in which the gaze detection unit 27 detects that the gaze of the user is directed to the AI image 51 displayed on the display screen 31 of the display unit 30. FIG. 12 is a diagram for describing the gaze detection unit 27.

As shown in FIG. 12, the gaze detection unit 27 acquires a face image of a face of the user captured by a camera C provided on an upper part of the display unit 30. The gaze detection unit 27 analyzes the acquired face image and detects the movement of a pupil E of the user to detect whether or not the gaze of the user is directed to the AI image 51 displayed on the display screen 31. Note that for the detection of the gaze, commonly used known technology can be used. The browsing detection unit 25 detects that the AI image 51 is browsed, for example, in a case in which the gaze of the user is directed to the AI image 51 for a predetermined time or longer. The browsing history 71 is recorded in the secondary storage unit 13 by the recording control unit 26 in the same manner as in the second embodiment.

In the third embodiment, it is possible to easily detect whether or not the user browses the AI image 51 by detecting the gaze of the user without any input operation by the user.

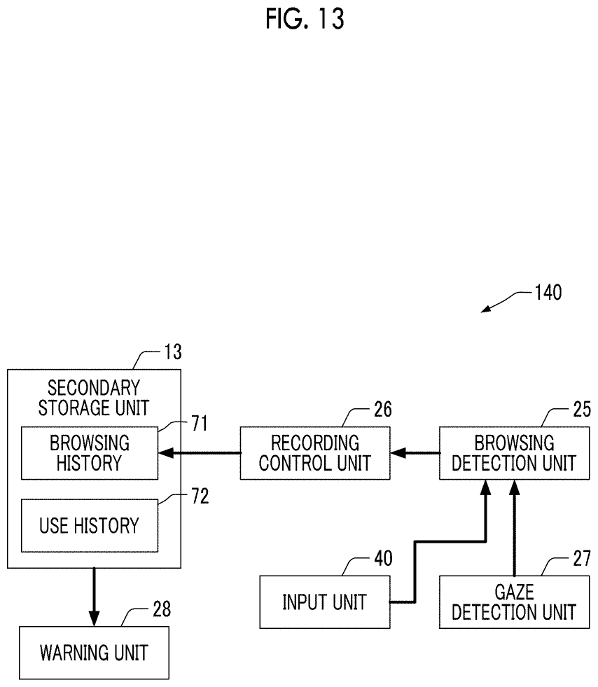

Next, a fourth embodiment of the present disclosure will be described. FIG. 13 is a functional block diagram of an image diagnosis support device 140 according to the fourth embodiment. In the image diagnosis support device 140 according to the fourth embodiment shown in FIG. 13, the CPU 11 of the image diagnosis support device 130 according to the third embodiment shown in FIG. 11 further has a function of a warning unit 28. Note that the function of the gaze detection unit 27 is the same as that of the third embodiment, the description thereof is omitted here.

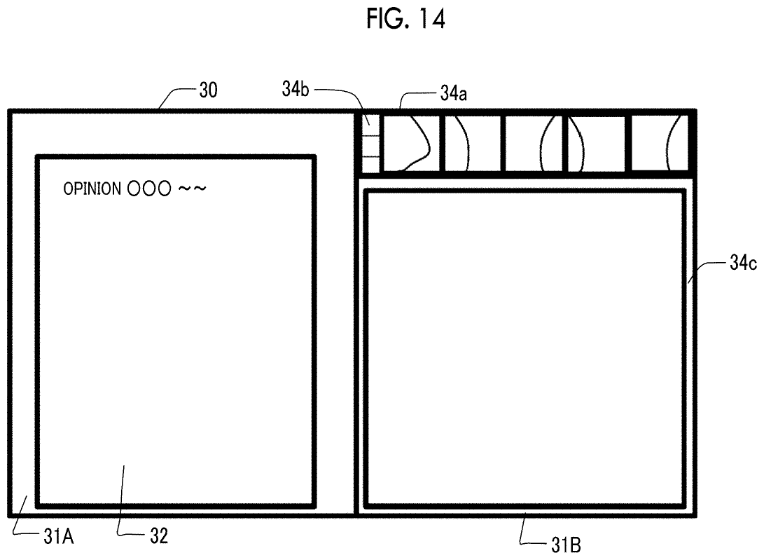

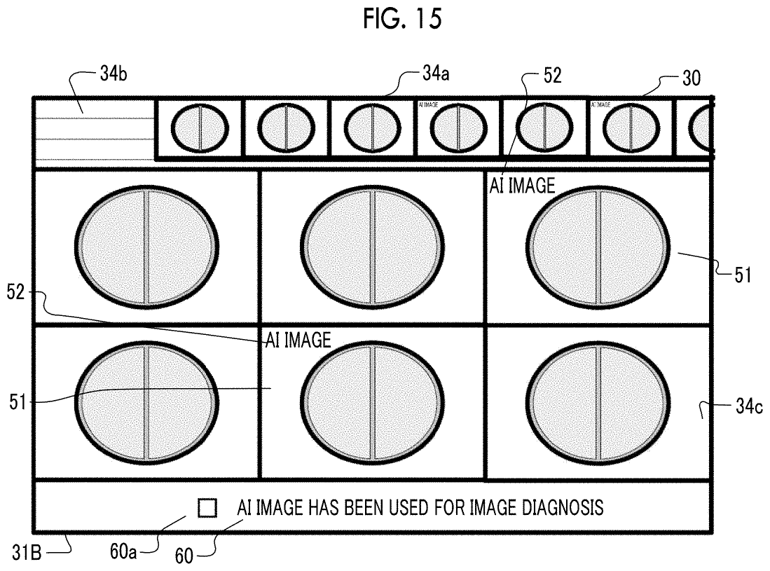

In the fourth embodiment, the recording control unit 26 performs a control of recording a use history 72 indicating that the AI image 51 has been used for the image diagnosis based on the operation of the user, in addition to the browsing history 71. First, in order to describe the operation of the user, the configuration of the display screen 31 of the display unit 30 according to the present embodiment will be described. FIG. 14 is a diagram showing an example of the display screen of the display unit according to the fourth embodiment, and FIG. 15 is a diagram showing an example of display on the second display screen of the display unit according to the fourth embodiment.

In the fourth embodiment, as shown in FIG. 14, the display unit 30 includes a first display screen 31A and a second display screen 31B. The display control unit 24 displays an interpretation report 32 in which the contents of the image diagnosis are recorded on the first display screen 31A, and displays the medical image on the second display screen 31B. The second display screen 31B functions as an image viewer on which the medical image is displayed. In the present embodiment, the display control unit 24 displays the contents displayed on the display screen 31 shown in FIG. 15 on the second display screen 31B.

On the second display screen 31B, a check box 60a is displayed below the image display region 34c, as shown in FIG. 15. The check box 60a is a use history input tool for inputting the use history 72 in a case in which the user uses the AI image 51 for the image diagnosis. Next to the check box 60a, text information 60, such as “AI image has been used for image diagnosis” is displayed to indicate the meaning of the check box 60a.

In the fourth embodiment, as shown in FIG. 13, in a case in which the user operates the mouse (input unit 40) to check the check box 60a, the recording control unit 26 stores the use history 72 indicating that the AI image 51 has been used for the image diagnosis in the secondary storage unit 13. Specifically, in the use history 72, the recording control unit 26 records the use history 72 in the secondary storage unit 13 in association with the image ID of the browsed AI image 51, that is, the AI image 51 displayed on the second display screen 31B.

In a case in which the interpretation report 32 related to the image diagnosis is created in a state in which the use history 72 is not present even though the browsing history 71 is present, the warning unit 28 issues a warning that the use history 72 is not present at least before creation of the interpretation report 32 is terminated. For example, the warning unit 28 causes the display control unit 24 to display warning information, such as “the use history of the AI image is not present”, on the first display screen 31A.

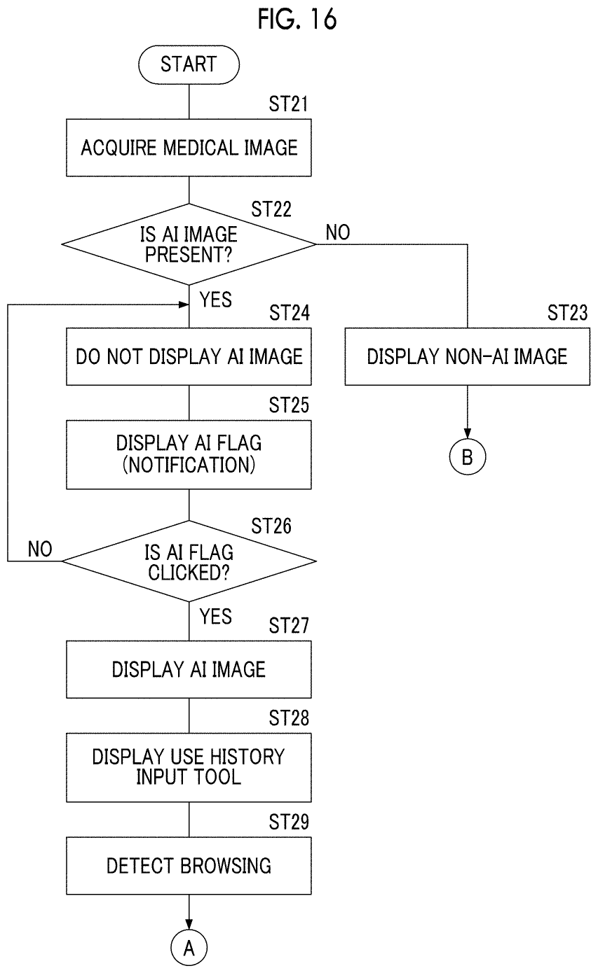

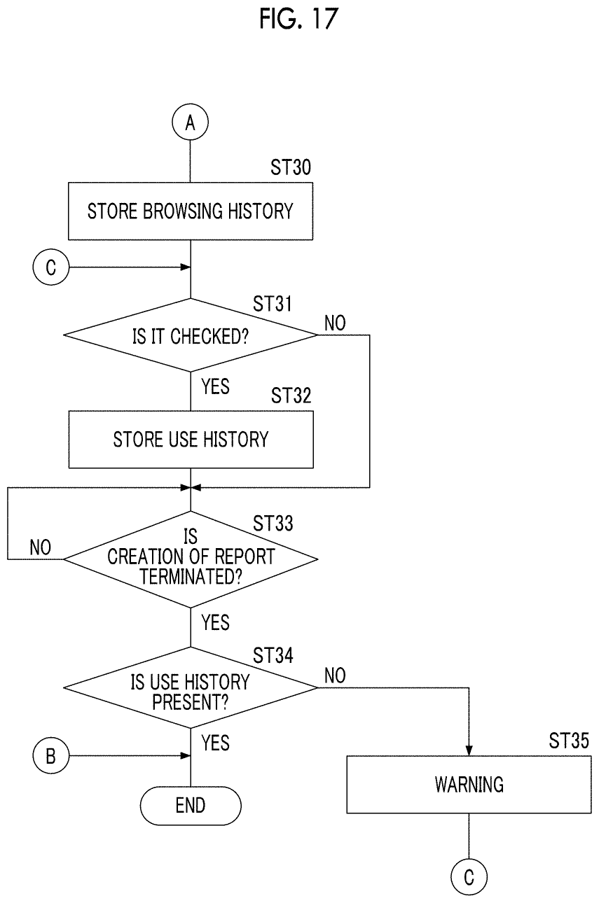

Then, processing performed in the present embodiment will be described. FIGS. 16 and 17 are flowcharts showing the processing performed in the fourth embodiment of the present disclosure.

First, the image acquisition unit 21 acquires the medical image in the same manner as in the first embodiment (step ST21). Then, the determination unit 22 determines whether or not the medical image acquired by the image acquisition unit 21 is the AI image 51 in the same manner as in the first embodiment (step ST22).

In a case in which step ST22 is denied (step ST22: NO), since the acquired medical image is not the AI image 51, that is, the non-AI image 50, the display control unit 24 displays the acquired medical image, that is, the non-AI image 50 on the second display screen 31B (step ST23), and the CPU 11 shifts the processing to B of FIG. 17 and terminates a series of the processing.

On the other hand, in a case in which step ST22 is affirmed (step ST22: YES), the display control unit 24 does not display the image contents while showing the presence of the acquired medical image, that is, the AI image 51 on the second display screen 31B (step ST24).

Then, the notification unit 23 causes the display control unit 24 to display the AI flag 52 (see reference numeral 52 in FIG. 9) indicating that the AI technology is applied to the AI image 51, which is not displayed (step ST25). Note that, in the present embodiment, displaying the AI flag 52 is an example of giving the notification that it is the AI image.

Then, the CPU 11 determines whether or not the AI flag 52 is clicked (step ST26). In a case in which step ST26 is denied (step ST26: NO), the CPU 11 shifts the processing to step ST24 and performs the subsequent processing. On the other hand, in a case in which step ST256 is affirmed (step ST26: YES), as shown in FIG. 16, the display control unit 24 displays the image contents of the AI image 51 (step ST27), and further displays the check box 60a (use history input tool) which is the use history input tool on the second display screen 31B (step ST28).

Then, the browsing detection unit 25 detects that the AI image 51 is browsed (step ST29). Then, as shown in FIG. 17, the recording control unit 26 records the browsing history 71 indicating that the AI image 51 is browsed in the secondary storage unit 13 based on the detection result of the browsing detection unit 25 (step ST30).

Then, the recording control unit 26 determines whether or not the check box 60a is checked (step ST31). In a case in which step ST31 is denied (step ST31: NO), the CPU 11 shifts the processing to step ST33. On the other hand, in a case in which step ST31 is affirmed (step ST31: YES), the recording control unit 26 records the use history 72 indicating that the AI image 51 has been used for the image diagnosis in the secondary storage unit 13 (step ST32).

Then, the CPU 11 determines whether or not the creation of the interpretation report 32 is terminated (step ST33). In a case in which step ST33 is denied (step ST33: NO), the CPU 11 repeatedly performs the processing of step ST33. On the other hand, in a case in which step ST33 is affirmed (step ST33: YES), the warning unit 28 determines whether or not the use history 72 is stored in the secondary storage unit 13 (step ST34). Note that, in the present embodiment, it is determined that the creation of the interpretation report 32 is terminated in a case in which the operation of closing the interpretation report 32 shown in FIG. 14 is performed. Specifically, the CPU 11 determines that the interpretation report 32 in closed in a case in which a cross mark (not shown) displayed on the upper right of the interpretation report 32 is clicked.

In a case in which step ST34 is affirmed (step ST34: YES), the CPU 11 terminates a series of the processing. On the other hand, in a case in which step ST34 is denied (step ST34: NO), the warning unit 28 issues the warning that the use history 72 is not present before the creation of the interpretation report 32 is terminated (step ST35), and the CPU 11 returns to processing of step ST31.

In the fourth embodiment, it is possible to prevent the creation of the interpretation report 32 related to the image diagnosis from being terminated in a state in which the use history 72 is not present even though the browsing history 71 is present.

Note that, in the fourth embodiment, the warning unit 28 displays the warning information, such as “the use history of the AI image is not present”, on the first display screen 31A, but the technology of the present disclosure is not limited to this. The warning unit 28 may display the warning information on the second display screen 31B instead of the first display screen 31A. In addition, the warning unit 28 may output the warning information by the voice.

In addition, in the fourth embodiment, in a case in which the creation of the interpretation report 32 is to be terminated in a state in which the use history 72 is not present even though the browsing history 71 is present, the warning unit 28 issues the warning that the use history 72 is not present, but the technology of the present disclosure is not limited to this. For example, the CPU 11 may perform processing in which the interpretation report 32 cannot be closed to prevent the creation of the interpretation report 32 from being terminated.

In addition, in the fourth embodiment, the first display screen 31A and the second display screen 31B are provided on the same display unit 30, but the technology of the present disclosure is not limited to this. In a case in which there are two display units 30, the display units 30 can display the first display screen 31A and the second display screen 31B, respectively.

In addition, in the embodiments described above, for example, as hardware structures of processing units that execute various pieces of processing, such as the image acquisition unit 21, the determination unit 22, the notification unit 23, the display control unit 24, the browsing detection unit 25, the recording control unit 26, the gaze detection unit 27, and the warning unit 28, it is possible to use various processors described below. As described above, various processors include, in addition to the CPU, which is a general-purpose processor that executes software (program) and functions as various processing units, a programmable logic device (PLD), which is a processor of which a circuit configuration can be changed after manufacture, such as a field programmable gate array (FPGA), and a dedicated electric circuit, which is a processor having a circuit configuration designed for exclusive use in order to execute specific processing, such as an application specific integrated circuit (ASIC).

One processing unit may be configured by one of various processors, or may be a combination of two or more processors of the same type or different types (for example, a combination of a plurality of FPGAs or a combination of the CPU and the FPGA). In addition, a plurality of the processing units may be configured by one processor.

As an example of configuring the plurality of processing units by one processor, first, as represented by a computer, such as a client and a server, there is a form in which one processor is configured by a combination of one or more CPUs and software and this processor functions as the plurality of processing units. Second, as represented by a system on chip (SoC) or the like, there is a form of using a processor that realizes the function of the whole system including the plurality of processing units with one integrated circuit (IC) chip. In this way, as the hardware structure, the various processing units are configured by using one or more of the various processors described above.

Further, as the hardware structure of these various processors, more specifically, it is possible to use an electrical circuit (circuitry) in which circuit elements such as semiconductor elements are combined.

What is claimed is:

1. An image diagnosis support device comprising:
a display control unit that displays a medical image acquired by imaging a subject on a display unit; and
a notification unit that gives, in a case in which an AI image, which is a medical image to which AI technology which is technology using artificial intelligence is applied, is displayed on the display unit as the medical image, a notification that the medical image displayed on the display unit is the AI image.

2. The image diagnosis support device according to claim 1,
wherein the AI image is a medical image newly generated separately from the medical image by applying the AI technology to the medical image.

3. The image diagnosis support device according to claim 1,
wherein the AI image is a medical image obtained by adding, to the medical image, an image analysis result obtained by performing an image analysis by using the AI technology based on the medical image.

4. The image diagnosis support device according to claim 1,
wherein the notification unit displays an AI flag indicating that the AI technology is applied to the AI image.

5. The image diagnosis support device according to claim 1, further comprising:
a determination unit that determines whether or not the medical image displayed on the display unit is the AI image.

6. The image diagnosis support device according to claim 5,
wherein accessory information of the medical image includes information indicating whether or not the AI technology is applied, and
the determination unit determines whether or not the medical image is the AI image based on the accessory information.

7. The image diagnosis support device according to claim 1, further comprising:
a browsing detection unit that detects whether or not a user browses the AI image; and
a recording control unit that performs a control of recording a browsing history indicating that the AI image is browsed, based on a detection result of the browsing detection unit.

8. The image diagnosis support device according to claim 7,
wherein the browsing detection unit detects that the AI image is browsed in a case in which the AI image, which has not displayed on the display unit, is displayed on the display unit.

9. The image diagnosis support device according to claim 8,
wherein the browsing detection unit detects that the AI image is browsed in a case in which a display instruction for displaying the AI image, which has not displayed, on the display unit is input.

10. The image diagnosis support device according to claim 7, further comprising:
a gaze detection unit that detects a gaze of the user,
wherein the browsing detection unit detects that the AI image is browsed in a case in which the gaze detection unit detects that the gaze of the user is directed to the AI image displayed on the display unit.

11. The image diagnosis support device according to claim 7,
wherein the recording control unit further performs a control of recording a use history indicating that the AI image is used for an image diagnosis, based on an operation of the user.

12. The image diagnosis support device according to claim 11, further comprising:
a warning unit that issues, in a case in which a report related to the image diagnosis is created in a state in which there is no use history even though there is the browsing history, a warning that there is no use history at least before creation of the report is complete.

13. An operation method of an image diagnosis support device, the method comprising:
displaying a medical image acquired by imaging a subject on a display unit; and
giving, in a case in which an AI image, which is a medical image to which AI technology which is technology using artificial intelligence is applied, is displayed on the display unit as the medical image, a notification that the medical image displayed on the display unit is the AI image.

14. A non-transitory storage medium storing a program that causes a computer to perform an image diagnosis support processing, the image diagnosis support processing comprising:
displaying a medical image acquired by imaging a subject on a display unit; and
giving, in a case in which an AI image, which is a medical image to which AI technology which is technology using artificial intelligence is applied, is displayed on the display unit as the medical image, a notification that the medical image displayed on the display unit is the AI image.
